(19) United States
(12) Patent Application Publication
(10) Pub. No.: US 2026/0272570 A1
(43) Pub. Date: Sep. 17, 2026

(54) ERGONOMIC ADJUSTMENTS FOR A PHYSICIAN CONSOLE

(71) Applicant: AURIS HEALTH, INC., Santa Clara, CA (US)

(72) Inventor: Fabien Y. Schmitt, San Jose, CA (US)

(21) Appl. No.: 19/668,697

(22) Filed: May 5, 2026

Related U.S. Application Data
(63) Continuation of application No. PCT/IB2024/061046, filed on Nov. 7, 2024.
(60) Provisional application No. 63/596,967, filed on Nov. 7, 2023.

(51) Int. Cl. A61B 34/00; A61B 34/37

(52) U.S. Cl. CPC A61B 34/25; A61B 34/37

(57) ABSTRACT

A physician console includes a base, a pillar coupled to the base, and a viewer coupled to the pillar. The viewer includes a viewer housing, a display within the viewer housing, and a control knob coupled to the viewer housing. Rotation of the control knob causes rotation of the viewer relative to the base.

### PRIORITY

This application is a continuation of International Application No. PCT/IB2024/061046, filed Nov. 7, 2024, entitled “ERGONOMIC ADJUSTMENTS FOR A PHYSICIAN CONSOLE,” which claims priority to U.S. Provisional Application No. 63/596,967, filed Nov. 7, 2023, entitled “ERGONOMIC ADJUSTMENTS FOR A PHYSICIAN CONSOLE,” the disclosures of which are incorporated by reference herein, in their entireties.

### BACKGROUND

Medical intervention has advanced over decades from purely open, manual surgical procedures, to more recently, minimally invasive and robotically assisted techniques. These advanced technologies can provide several benefits, such as reduced trauma and recovery times, as well improved dexterity and targeting precision. Robotic systems may employ a console that provides an interface for a physician to control a surgical robot as well as observe a surgical site. Physicians may be seated at these consoles for prolonged periods of time.

### BRIEF DESCRIPTION OF DRAWINGS

The subject technology will be understood from the detailed description of certain examples taken in conjunction with the accompanying drawings, in which:

FIG. 1 shows an example of a surgical system, in accordance with some embodiments.

FIG. 2 shows an example of a physician console, in accordance with some embodiments.

FIG. 3 shows an example of ergonomic adjustments of a physician console, in accordance with some embodiments.

FIG. 4 shows an example of ergonomic adjustments of a physician console, in accordance with some embodiments.

The accompanying drawings incorporated in and forming a part of the specification illustrate several aspects of the subject technology, and together with the description serve to explain the principles of the technology. It is understood, however, that the technology is not limited to the precise configurations shown, and that various embodiments of the technology may be carried out in a variety of other ways, including those not necessarily depicted in the drawings.

### DETAILED DESCRIPTION

The detailed description below provides particular configurations of the subject technology but is not intended to represent the only possible configurations in which the technology may be practiced. The detailed description, including the appended drawings, includes various specific details to aid in understanding the technology, but it will be clear that principles of the technology are not limited to all of the specific details set forth herein and may be practiced in different configurations. Other features, embodiments, and advantages of the technology will become apparent to those skilled in the art from the following detailed description, and as will be realized, the technology described herein is capable of other different and obvious configurations, all without departing from the principles of the technology and the scope of the teachings provided by this disclosure. Further, the various features and configurations described herein should not be viewed in isolation relative to each other, and it will be apparent to those skilled in the art that many different features and configurations described herein may be combined with each other in a variety of ways that are not expressly set forth below. Accordingly, the drawings and descriptions provided herein should be regarded as illustrative in nature and not restrictive.

As noted above, robotic surgical systems may employ a console that a physician may use to interact with the system. A physician console can include one or more input devices, such as hand controls and foot pedals, where the physician can provide commands that control a surgical robot. The physician console can also include a viewer that houses a display for the surgeon to view the surgical site. The physician may be seated at the surgeon console and place their head in the display while manipulating the input devices with their hands and feet. Such systems may provide an instinctive interface with which the surgeon can manipulate a robot to perform procedural tasks, while observing the surgical site through the real-time video feed provided at the viewer. However, prolonged usage of these systems can lead to surgeon discomfort and fatigue, limiting adoption of these systems and compromising patient care.

Among other things, systems and methods disclosed herein can provide for improved ergonomics in a surgeon console and more instinctive techniques for making ergonomic adjustments in a console. These and other features are discussed in more detail with reference to the examples shown in the figures described below. However, those skilled in the art will readily that the subject technology and the innovative concepts disclosed herein may be practiced in a variety of different configurations. Further, a variety of different problems may be addressed and advantages obtained with the subject technology. Accordingly, the above description and the detailed description that follows is provided for explanatory purposes but should not be construed as limiting.

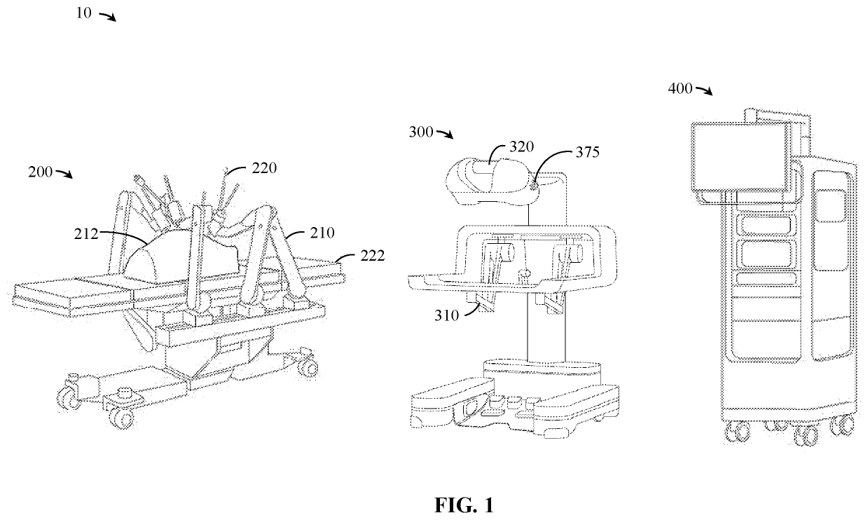

FIG. 1 illustrates an example of a surgical system, in accordance with some embodiments. As illustrated, the surgical system 10 includes a robot 200, a physician console 300, and a tower 400. These components can operate together to facilitate a surgical procedure and may be coupled together physically, communicatively, and/or operatively.

The robot 200 can be part of a patient-side system. As seen in FIG. 1, robot 200 is configured to interact with a patient 212, which may be positioned on a table 222. Robot 200 can include one or more robotic manipulators 210, which can manipulate one or more instruments 220. The robotic manipulators 210 can move or be actuated responsive to commands received at the physician console 300, the tower 400, and/or at the robot 200 itself. The robotic manipulators 210 can each include a robotic arm having a series of links connected by a series of joints.

The physician console 300 can be part of a physician-side system, where a physician interacts with the surgical system 10. The physician console 300 provides an interface for controlling robot 200 and/or observing information captured by the patient-side system. Physician console 300 can include one or more input devices 310, which can receive inputs from a physician for controlling robot 200. Alternatively, or in combination, physician console 300 can include one or more output devices 320, which can output information captured at the patient side system. As seen in FIG. 1, physician console may further include a control knob 375, which can receive inputs for adjusting ergonomic positioning of the console. Physician console 300 may be located in the same operating room as the robot 200 or may be located at a remote site away from the operating room.

The tower 400 can provide supporting functionality for surgical system. For example, the tower can provide energy delivery, fluidics, vision processing, or light to the robot 200 or instruments 220.

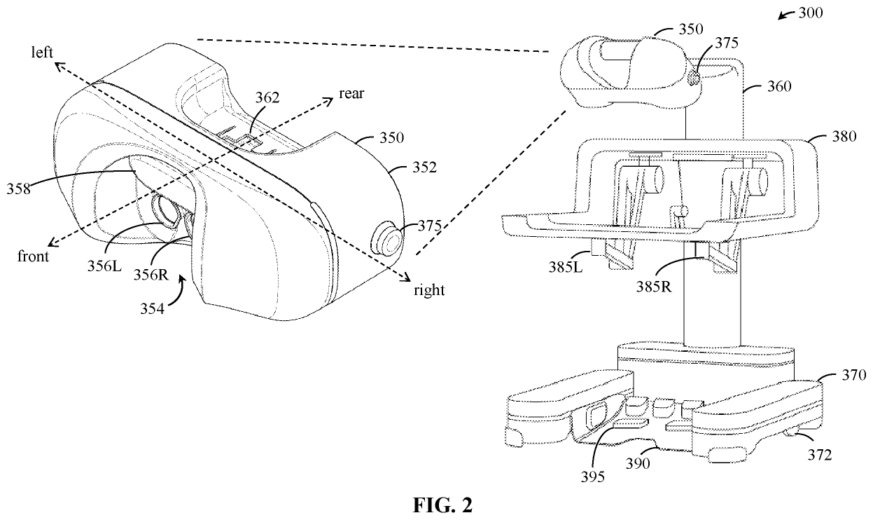

Referring to FIG. 2, physician console 300 includes a viewer 350, which can provide an imaging feed for viewing by a physician. For example, the viewer can be configured to display a real-time video captured by an instrument 220 (e.g., an endoscope) at the surgical site.

As seen in FIG. 2, viewer 350 is coupled to a pillar 360, which provides a vertical support structure for holding the viewer. Pillar 360 can be coupled to a base 370, which can be configured to rest on the floor and provide a stable base for supporting the console. Base 370 may include wheels 372 to assist with moving and positioning the console as desired.

As seen in FIG. 2, physician console 300 further includes an armrest 380, which is also coupled to pillar 360. Console 300 can include one or more hand input devices (HIDs), which can be coupled to the armrest 380 or the pillar 360 for support. As illustrated, console 300 includes a pair of HIDs coupled to the armrest 380, including a left HID 385L and a right HID 385R. Left and right HIDs can be configured to be held by left and right hands of a physician and manipulated to provide control inputs to the surgical robot 200 for manipulating instruments 220, such as moving or positioning instruments in space, or actuating functions of instruments such as grasping or cutting tissue. As seen in FIG. 2, physician console 300 may also include a footboard assembly 390 having one or more foot pedals 395. Foot pedals 395 can be configured to be operated by a physician positioned at the console to perform additional tasks, such as applying energy, firing a stapler, or selecting different instruments for active control.

With continued reference to FIG. 2, an enlarged view of the viewer 350 is shown on the left. Viewer 350 includes a housing 352 having a front side, a rear side, a left side, and a right side. Front side of the housing 352 can include a viewing window through which a user can view a display housed in the viewer. Front side of the housing 352 may further include a face region 354 for a physician to place their head in the viewer to view the display. Face region 354 can include a cutaway region of the front side of the housing that is ergonomically shaped to receive the user's head. Face region 354 may include a forehead rest 358 that include a cushion for enhanced comfort when the physician's head is rested against the viewer. The display may be configured as any suitable display for displaying images or video to the physician. In the illustrated example, the display housed within the viewer is a three-dimensional, stereoscopic display having a left eye display 356L and a right eye display 356R for presenting a stereoscopic imaging feed to the physician.

As seen in FIG. 2, a viewer housing 352 can include a mounting portion 362 for mounting the viewer to the pillar 360. Mounting portion 362 can be positioned on a rear side of the viewer housing and can include a surrounding cutout region to accommodate adjustments of the orientation of the viewer with respect to the pillar.

Viewer 350 includes a control knob 375 coupled to the viewer housing 350. Control knob 375 can be operable by a physician to adjust a positioning of the viewer relative to the base 370 or the pillar 360. Positioning the control knob 375 on the housing of the viewer may facilitate improved immersion and improved usability by allowing the physician to easily and instinctively control an ergonomic positioning of the viewer without requiring the user to remove their head from the viewer. Control knob 375 may be positioned on the left side or the right side of the viewer housing to facilitate instinctive adjustments with the user's left or right hand, and in the illustrated example, the control knob 375 is positioned on the right side of the housing as more users are likely to be right-handed.

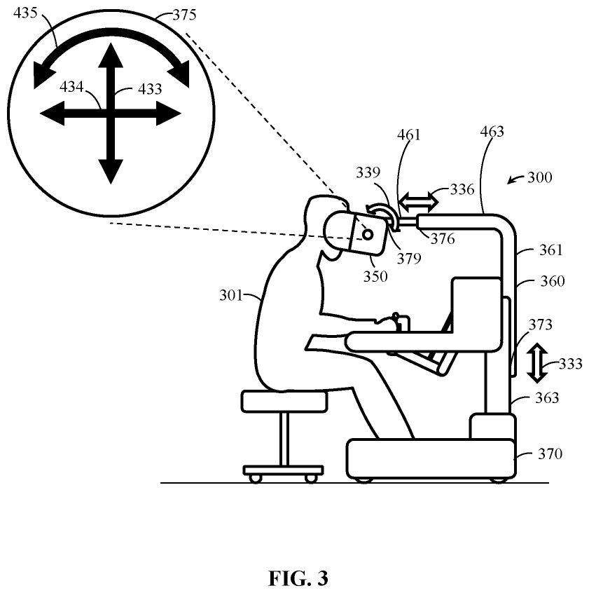

FIG. 3 shows a side profile view of the physician console 300 with a physician 301 seated at the console and positioned with their head in the viewer 350. FIG. 3 illustrates various degrees of freedom that may be employed in the console and controlled by control knob 375 to adjust an ergonomic positioning of the console, in accordance with some embodiments.

Referring to FIG. 3, console 300 can include one or more of a height degree of freedom 333, a depth degree of freedom 336, and a rotation degree of freedom 339. One or more of these degrees of freedom may be controlled by control knob 375 to adjust a positioning of the viewer in the corresponding degree of freedom. It is contemplated that any grouping of one or more of these degrees of freedom may be controlled by any grouping of one or more movements or inputs to the control knob. In some configurations, a single control knob can be operated via different movements to control all of the degrees of freedom.

With continued reference to FIG. 3, the height degree of freedom 333 may be controlled by a height joint 373. Height joint 373 can be positioned in a vertical portion of the pillar 360 and can connect an upper vertical portion 361 of the pillar with a lower vertical portion 363 of the pillar. Operation of the control knob along a vertical axis 433 can actuate the height joint 373 to translate the viewer vertically. For example, pushing or moving the control knob 375 upward can provide a signal that causes an actuator (e.g., a motor) to raise the upper vertical portion 361 relative to the lower vertical portion 363. Pushing or moving the control knob 375 in the opposite direction (downward) can provide a signal that causes an actuator (e.g., a motor) to move the upper vertical portion 361 relative to the lower vertical portion 363 in an opposite direction (lower the upper vertical portion 361). As the viewer 350 is mounted to the pillar, this movement can raise or lower the position of the viewer relative to the base accordingly.

The depth degree of freedom 336 may be controlled by a depth joint 376. Depth joint 376 can be positioned in a horizontal portion of the pillar 360 and can connect a proximal horizontal portion 461 of the pillar with a distal horizontal portion 463 of the pillar. Operation of the control knob along a horizontal axis 434 can actuate the depth joint 376 to translate the viewer horizontally. For example, pushing or moving the control knob 375 rearward (with respect to the physician positioning) can provide a signal that causes an actuator (e.g., a motor) to retract the proximal horizontal portion 461 relative to the distal horizontal portion 463. Pushing or moving the control knob 375 in the opposite direction (forward) can provide a signal that causes an actuator (e.g., a motor) to move the proximal horizontal portion 461 relative to the distal horizontal portion 463 in an opposite direction (advance proximal horizontal portion 461 forward). As the viewer 350 is mounted to the pillar, this movement can advance or retract the position of the viewer relative to the pillar accordingly.

The rotational degree of freedom 339 (orientation) may be controlled by a rotational joint 379. Rotation joint 379 can be positioned in a horizontal portion of the pillar 360 and can connect the viewer housing to the pillar. Operation of the control knob along a rotational axis 435 (rotation) can actuate the rotational joint 379 to rotate the orientation of the viewer. For example, rotating the control knob 375 counterclockwise can provide a signal that causes an actuator (e.g., a motor) to rotate the viewer housing clockwise relative to the base 370 or relative to the pillar 360. Rotating the control knob 375 in the opposite direction (clockwise) can provide a signal that causes an actuator (e.g., a motor) to rotate the viewer housing in an opposite direction (clockwise).

In some configurations, rotation of the control knob can cause rotation of the viewer alone. In some configuration, rotation of the control knob can cause a corresponding translation of the viewer to account for ergonomics of a physician seated at the console.

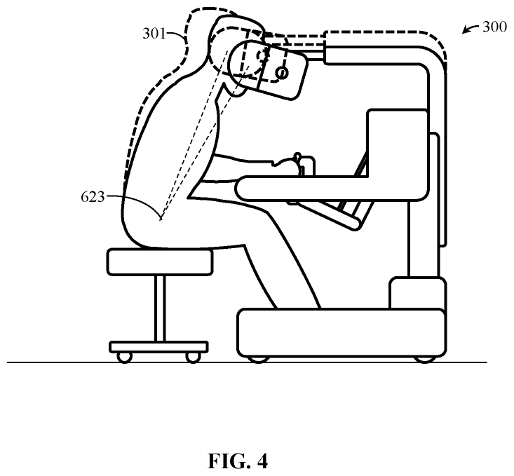

FIG. 4 illustrates an example of a rotation adjustment, where a corresponding translation (location adjustment) of the viewer is actuated in concert with the rotation adjustment to maintain a virtual pelvis location 623 of the physician 301. Solid lines show the physician 301 and console 300 before the rotation adjustment is applied, while dotted lines show the after the rotation adjustment is applied. Upon the physician operating the rotation adjustment (e.g., rotating control knob 375), the console automatically applies a corresponding translation of the viewer to move the viewer about the virtual pelvis location 623. This makes it more instinctive for the physician to remain seated at the console and adjust the orientation of the viewer without having to readjust their seating position.

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

Example 1: A physician console, comprising: a base; a pillar coupled to the base; and a viewer coupled to the pillar, the viewer comprising: a viewer housing; a display within the viewer housing; and a control knob coupled to the viewer housing, wherein rotation of the control knob causes rotation of the viewer relative to the base.

Example 2: The physician console of Example 1, wherein rotation of the control knob in a clockwise direction causes rotation of the viewer in a clockwise direction and rotation of the control knob in a counterclockwise direction causes rotation of the viewer in a counterclockwise direction.

Example 3: The physician console of Example 1 or Example 2, wherein the control knob is further operable to cause translation of the viewer relative to the base.

Example 4: The physician console of any one of Examples 1-3, wherein pushing the control knob in a forward direction advances the viewer relative to the pillar and pushing the control knob in a rearward direction retracts the viewer relative to the pillar.

Example 5: The physician console of any one of Examples 1-4, wherein pushing the control knob in an upward direction raises the viewer relative to the base and pushing the control knob in a downward direction lowers the viewer relative to the base.

Example 6: The physician console of any one of Examples 1-5, wherein the pillar comprises a height joint operable to raise or lower the viewer relative to the base, and wherein the control knob is operable to actuate the height joint of the pillar.

Example 7: The physician console of any one of Examples 1-6, wherein the pillar comprises a depth joint operable to advance or retract the viewer relative to the pillar, and wherein the control knob is operable to actuate the depth joint of the pillar.

Example 8: The physician console of any one of Examples 1-7, wherein the viewer is coupled to the pillar at a rotation joint, wherein rotation of the control knob causes actuation of the rotation joint.

Example 9: The physician console of any one of Examples 1-8, wherein rotation of the control knob further causes translation of the viewer relative to the base to move the viewer about a virtual pelvis.

Example 10: The physician console of any one of Examples 1-9, wherein the viewer housing comprises a front side, a left side adjacent the front side, and a right side adjacent the front side and opposing the front side, wherein the front side comprises a viewing window for the display, and the control knob is positioned on the left side or the right side.

Example 11: A physician console, comprising: a base; a pillar coupled to the base; and a viewer coupled to the pillar, the viewer comprising: a viewer housing; a display within the viewer housing; and a control knob coupled to the viewer housing, wherein the control knob is operable to advance or retract the viewer relative to the pillar.

Example 12: The physician console of Example 11, wherein rotation of the control knob in a clockwise direction causes rotation of the viewer in a clockwise direction and rotation of the control knob in a counterclockwise direction causes rotation of the viewer in a counterclockwise direction.

Example 13: The physician console of Example 11 or Example 12, wherein the control knob is further operable to cause translation of the viewer relative to the base.

Example 14: The physician console of any one of Examples 11-13, wherein pushing the control knob in a forward direction advances the viewer relative to the pillar and pushing the control knob in a rearward direction retracts the viewer relative to the pillar.

Example 15: The physician console of any one of Examples 11-14, wherein pushing the control knob in an upward direction raises the viewer relative to the base and pushing the control knob in a downward direction lowers the viewer relative to the base.

Example 16: The physician console of any one of Examples 11-15, wherein the pillar comprises a height joint operable to raise or lower the viewer relative to the base, and wherein the control knob is operable to actuate the height joint of the pillar.

Example 17: The physician console of any one of Examples 11-16, wherein the pillar comprises a depth joint operable to advance or retract the viewer relative to the pillar, and wherein the control knob is operable to actuate the depth joint of the pillar.

Example 18: The physician console of any one of Examples 11-17, wherein the viewer is coupled to the pillar at a rotation joint, wherein rotation of the control knob causes actuation of the rotation joint.

Example 19: The physician console of any one of Examples 11-18, wherein rotation of the control knob further causes translation of the viewer relative to the base to move the viewer about a virtual pelvis.

Example 20: The physician console of any one of Examples 11-19, wherein the viewer housing comprises a front side, a left side adjacent the front side, and a right side adjacent the front side and opposing the front side, wherein the front side comprises a viewing window for the display, and the control knob is positioned on the left side or the right side.

The forgoing description provides many specific configurations and details to aid in understanding the principles of the technology described herein, but it will be understood that the technology can be practiced in various configurations. Accordingly, the claims are not intended to be limited to only the specific details and configurations described herein but are instead to be afforded their full scope, consistent with their language and all legal equivalents.

1. A physician console, comprising:
a base;
a pillar coupled to the base; and
a viewer coupled to the pillar, the viewer comprising:
a viewer housing;
a display within the viewer housing; and
a control knob coupled to the viewer housing, wherein rotation of the control knob causes rotation of the viewer relative to the base.

2. The physician console of claim 1, wherein rotation of the control knob in a clockwise direction causes rotation of the viewer in a clockwise direction and rotation of the control knob in a counterclockwise direction causes rotation of the viewer in a counterclockwise direction.

3. The physician console of claim 1 wherein the control knob is further operable to cause translation of the viewer relative to the base.

4. The physician console of claim 1, wherein pushing the control knob in a forward direction advances the viewer relative to the pillar and pushing the control knob in a rearward direction retracts the viewer relative to the pillar.

5. The physician console of claim 1, wherein pushing the control knob in an upward direction raises the viewer relative to the base and pushing the control knob in a downward direction lowers the viewer relative to the base.

6. The physician console of claim 1, wherein the pillar comprises a height joint operable to raise or lower the viewer relative to the base, and wherein the control knob is operable to actuate the height joint of the pillar.

7. The physician console of claim 1, wherein the pillar comprises a depth joint operable to advance or retract the viewer relative to the pillar, and wherein the control knob is operable to actuate the depth joint of the pillar.

8. The physician console of claim 1, wherein the viewer is coupled to the pillar at a rotation joint, wherein rotation of the control knob causes actuation of the rotation joint.

9. The physician console of claim 1, wherein rotation of the control knob further causes translation of the viewer relative to the base to move the viewer about a virtual pelvis.

10. The physician console of claim 1, wherein the viewer housing comprises a front side, a left side adjacent the front side, and a right side adjacent the front side and opposing the front side, wherein the front side comprises a viewing window for the display, and the control knob is positioned on the left side or the right side.

11. A physician console, comprising:
a base;
a pillar coupled to the base; and
a viewer coupled to the pillar, the viewer comprising:
a viewer housing;
a display within the viewer housing; and
a control knob coupled to the viewer housing, wherein the control knob is operable to advance or retract the viewer relative to the pillar.

12. The physician console of claim 11, wherein rotation of the control knob in a clockwise direction causes rotation of the viewer in a clockwise direction and rotation of the control knob in a counterclockwise direction causes rotation of the viewer in a counterclockwise direction.

13. The physician console of claim 11 wherein the control knob is further operable to cause translation of the viewer relative to the base.

14. The physician console of claim 11, wherein pushing the control knob in a forward direction advances the viewer relative to the pillar and pushing the control knob in a rearward direction retracts the viewer relative to the pillar.

15. The physician console of claim 11, wherein pushing the control knob in an upward direction raises the viewer relative to the base and pushing the control knob in a downward direction lowers the viewer relative to the base.

16. The physician console of claim 11, wherein the pillar comprises a height joint operable to raise or lower the viewer relative to the base, and wherein the control knob is operable to actuate the height joint of the pillar.

17. The physician console of claim 11, wherein the pillar comprises a depth joint operable to advance or retract the viewer relative to the pillar, and wherein the control knob is operable to actuate the depth joint of the pillar.

18. The physician console of claim 11, wherein the viewer is coupled to the pillar at a rotation joint, wherein rotation of the control knob causes actuation of the rotation joint.

19. The physician console of claim 11, wherein rotation of the control knob further causes translation of the viewer relative to the base to move the viewer about a virtual pelvis.

20. The physician console of claim 11, wherein the viewer housing comprises a front side, a left side adjacent the front side, and a right side adjacent the front side and opposing the front side, wherein the front side comprises a viewing window for the display, and the control knob is positioned on the left side or the right side.
